(19) United States
(12) Patent Application Publication
(10) Pub. No.: US 2026/0337165 A1
(43) Pub. Date: Oct. 29, 2026

(54) METHODS FOR DEPOLYMERIZING POLYAMIDES

(71) Applicant: Northwestern University, Evanston, IL (US)

(72) Inventors: Liwei Ye, Evanston, IL (US); Yosi Kratish, Evanston, IL (US); Tobin J. Marks, Evanston, IL (US); Qi Zhao, Evanston, IL (US)

(21) Appl. No.: 19/483,844

(22) PCT Filed: May 14, 2024
(86) PCT No.: PCT/US2024/029193

§ 371 Date: Nov. 12, 2025

Related U.S. Application Data
(60) Provisional application No. 63/466,775, filed on May 16, 2023.

(51) Int. Cl. C07D 223/10

(52) U.S. Cl. CPC C07D 223/10

(57) ABSTRACT

Provided are methods for depolymerizing polyamides. In embodiments, such a method comprises combining a polyamide and an alkali metal catalyst to depolymerize the polyamide to a product, wherein the alkali metal catalyst is selected from those having a formula MXRₙ, wherein M is an alkali metal; X is H, C, N, Si, or Sn; R is independently selected from hydrogen, alkyl, silyl, and aryl; and n is from 0 to 2.

### CROSS-REFERENCE TO RELATED APPLICATIONS

The present application claims priority to U.S. provisional patent application No. 63/466,775 that was filed May 16, 2023, the entire contents of which are incorporated herein by reference.

### REFERENCE TO GOVERNMENT RIGHTS

This invention was made with government support under grant numbers DE-SC0022290 and DE-FG02-03ER15457 awarded by the Department of Energy. The government has certain rights in the invention.

### BACKGROUND

Our modern society is heavily dependent on plastics-based materials, as evident by the steady growth in their production. Plastics are extremely ubiquitous, versatile, and low-cost polymeric materials that have dramatically enhanced the quality of human life for more than a century. Currently, plastics are being produced worldwide at an annual rate of 450 million tons year, which is projected to double by 2045. One class of commonly used plastics for applications requiring the material/product to withstand harsh mechanical and environmental conditions are engineering plastics such as polyamides.

Nylon-6 was one of the first synthetic fibers discovered and developed by Schlack in 1938. Nylon-6 is a thermoplastic polyamide produced industrially by water-assisted ring-opening polymerization (ROP) of ε-caprolactam on an 8.9 million tons annual scale, with the market size expected to reach $21.5 billion in 2026. Nylons as non-degradable plastics substantially contribute to the pollution in oceans and landfills, reflecting their superior chemical robustness and paucity of effective recycling technologies. Indeed, it is estimated that by 2050, plastic waste will outweigh fish in the ocean, with Nylon-6 contributing ca. 10% of ocean plastic pollution as discarded or lost fishing nets (so-called “ghost nets”). This amounts to more than 600,000 tons of abandoned fishing nets per year.

### SUMMARY

Provided are methods for depolymerizing polyamides, e.g., Nylon-6, using certain alkali metal catalysts which are further described below. The Example, below, describes experimental results demonstrating that mild conditions may be used in the methods (e.g., temperatures as low as 220° C. and reaction times of less than a few hours, even as little as a few minutes) along with low catalyst loadings to afford monomers of the polyamides, e.g., ε-caprolactam, in surprisingly high yields (e.g., about 100%). Moreover, the methods may be carried out either under vacuum or at higher pressures, including under an inert atmosphere. No solvents or other additives are required. The methods may be carried out continuously, for extended periods of time, without a reduction in reaction rate. The alkali metal catalysts, e.g., in a reaction mixture with a polyamide, are also encompassed by the present disclosure.

In an embodiment, a method for depolymerizing a polyamide comprises combining a polyamide and an alkali metal catalyst to depolymerize the polyamide to a product, wherein the alkali metal catalyst is selected from those having a formula MXRₙ, wherein M is an alkali metal; X is H, C, N, Si, or Sn; R is independently selected from hydrogen, alkyl, silyl, and aryl; and n is from 0 to 2.

Other principal features and advantages of the disclosure will become apparent to those skilled in the art upon review of the following drawings, the detailed description, and the appended claims.

### BRIEF DESCRIPTION OF THE DRAWINGS

Illustrative embodiments of the disclosure will hereafter be described with reference to the accompanying drawings.

FIGS. 1A-1D show examples of Nylon-6 chemical recycling technologies to ε-caprolactam monomer, including using existing techniques (FIGS. 1A-1C) and using an illustrative embodiment according to the present disclosure (FIG. 1D).

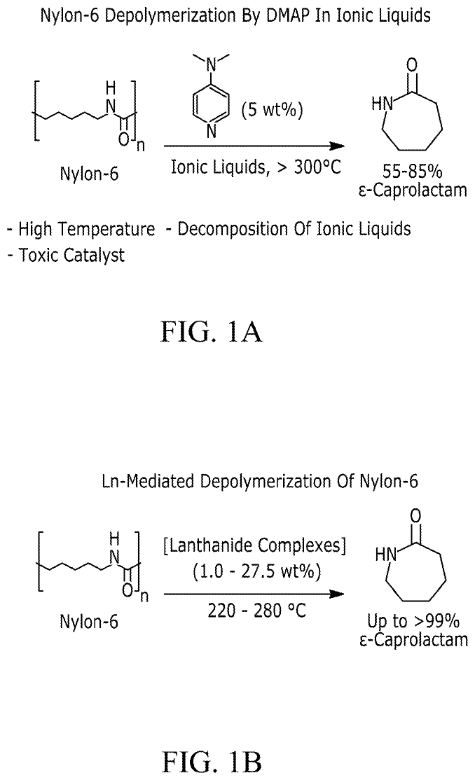

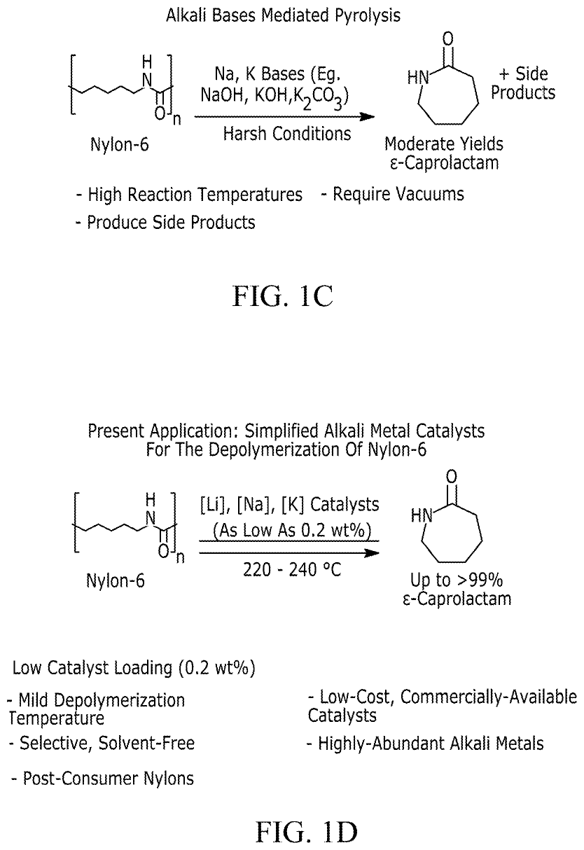

### DETAILED DESCRIPTION

Provided are methods for depolymerizing polyamides. In an embodiment, such a method comprises combining a polyamide and a superbase alkali metal catalyst under conditions to depolymerize the polyamide.

The polyamide to be depolymerized by the present methods is a polymer composed of monomers covalently bound into an extended chain via amide linking groups. The term “monomer” refers to the chemical reactant that is incorporated to form the extended chain and the amide linking groups during a polymerization reaction. The polyamide may be an aliphatic polyamide (i.e., not comprising aromatic rings) or an aromatic polyamide (i.e., comprising aromatic rings). The polyamide may be a homopolymer (i.e., formed from a single type of monomer) or a heteropolymer (i.e., formed from more than one type of monomer, e.g., two; “heteropolymers” may be referred to as copolymers). The term “type” refers to a chemical formula such that a single type means the same chemical formula and different type means different chemical formulas.

The polyamide may be one which is formed by ring-opening polymerization of a cyclic amide (i.e., lactam). In such embodiments, the monomer is a cyclic amide and the polymerization reaction is ring-opening polymerization. Illustrative cyclic amides include 2-pyrrolidone, 2-piperidone, ε-caprolactam, enantholactam, capryllactam, pelargolactam, azacycloundecan-2-one, and azacyclotridecan-2-one.

The polyamide may be one which is formed from condensation of an amine (including a diamine) with an acid (including a diacid). The amine and the acid may be provided on a single chemical compound such as 11-aminoundecanoic acid or @-aminolauric acid). Alternatively, the amine and the acid may be provided on two different chemical compounds such as hexamethylene diamine and adipic acid. In these embodiments, the monomer is the single chemical compound (with the amine and acid groups) or the two chemical compounds (the diamine and the diacid), and the polymerization reaction is condensation.

The polyamide may be identified by reference to the monomers (e.g., cyclic amide, diamine, diacid) used to form the polyamide, recognizing that the chemical form of these monomers may be modified by the ensuing polymerization reaction that provides the amide linking groups of the polyamide.

Illustrative polyamides to be depolymerized in the present methods include poly(2-pyrrolidinone) (Nylon-4), poly(2-piperidone) (Nylon-5), poly(hexano-6-lactam) (Nylon-6), polyenanthamide (Nylon-7), polycapryllactam (Nylon-8), poly(9-aminononanoic acid (Nylon-9), poly(10-aminodecanoic acid) (Nylon-10), poly(11-aminoundecanoic acid) (Nylon-11), poly(dodecano-12-lactam) (Nylon-12), poly[imino(1,6-dioxo hexamethylene)imino hexamethylene] (Nylon-66), and poly[imino(1,6-dioxohexamethylene) iminotetramethylene] (Nylon-46).

The present methods may be used to depolymerize any of the disclosed polyamides. The depolymerization process deconstructs the polyamide into a product. In embodiments, the depolymerization process provides a monomer (e.g., a cyclic amide) from which the polyamide was formed, i.e., the product comprises (or consists of) the monomer of the polyamide. Throughout this disclosure, the terms “a monomer” and “monomer” encompass both a single type of monomer and multiple, different types of monomers, i.e., depending upon the particular polyamide. In such embodiments, this monomer may be recovered and used to reform the polyamide. This is by contrast to depolymerization processes which do not provide a monomer, but rather a product comprising a chemical compound which cannot reform the polyamide absent additional steps to convert the chemical compound to the monomer. However, the present methods encompass both types of depolymerization processes, i.e., those that provide the monomer of the polyamide and those that provide a different type of product

In embodiments, the methods depolymerize Nylon-6 to produce ε-caprolactam. In embodiments, the methods depolymerize Nylon-4 to produce 2-pyrrolidone. In embodiments, the methods depolymerize Nylon-5 to produce 2-pyrrolidone. In embodiments, the methods depolymerize Nylon-7 to produce enantholactam. In embodiments, the methods depolymerize Nylon-8 to produce capryllactam. In embodiments, the methods depolymerize Nylon-9 to produce pelargolactam. In embodiments, the methods depolymerize Nylon-10 to produce azacycloundecan-2-one. In embodiments, the methods depolymerize Nylon-11 to produce azacyclotridecan-2-one. In embodiments, the methods depolymerize Nylon-12 to produce @-aminolauric acid.

A single type of polyamide or multiple, different types of polyamides may be used (i.e., a blend of different types of polyamides).

The alkali metal catalysts used in the present methods include those having Formula I, MXRₙ, wherein M is an alkali metal; X is H, C, N, Si, or Sn; R is independently selected from hydrogen, alkyl, silyl, and aryl; and the value of n (which may be zero) depends upon the selected X. In the present disclosure, such alkali metal catalysts may be referred to as “superbases” and like terms. It is understood that Formula I encompasses embodiments in which X is an atom of the selected R group. For example, when X is C, this carbon may be an atom of the selected R group, e.g., a carbon of an alkyl group as defined below. In other words, X and R together may form an alkyl group, e.g., an unsubstituted linear alkyl group or a substituted branched alkyl group. This means that when X is C, an additional hydrogen(s) may be bound to the carbon. As another example, when X is N, this nitrogen may be a heteroatom of the selected R group, e.g., a nitrogen of a cycloalkyl group as defined below. In other words, X and R together may form a nitrogen-substituted cycloalkyl group. This means that when X is N, more than one atom (e.g., two carbons) of the R group may be bound to the nitrogen.

Formula I does not encompass alkali metal oxides, alkali metal hydroxides, alkali metal carbonates, and alkali metal carboxylates. Thus, the present catalysts are not any of these compounds.

In Formula I and its related formulas, various alkali metals may be used. Illustrative alkali metals include Li, Na, K, Cs, and a mixture thereof.

In Formula I and its related formulas, “alkyl group” refers to a linear, branched, or cyclic alkyl group in which the number of carbons may range from, e.g., 1 to 8, 1 to 6, 1 to 4, 1 to 3, or 1 to 2. A cyclic alkyl group may be referred to as a cycloalkyl group. The alkyl group may be unsubstituted, by which it is meant the alkyl group contains no heteroatoms. An unsubstituted alkyl group encompasses an alkyl group in which one or more bonds to a carbon(s) or hydrogen(s) are replaced by a bond to an unsubstituted aromatic ring, e.g. benzyl. The alkyl group may be substituted, by which it is meant an unsubstituted alkyl group in which one or more bonds to a carbon(s) or hydrogen(s) are replaced by a bond to non-hydrogen and non-carbon atoms.

In Formula I and its related formulas, “aryl group” refers to a monocyclic aryl group having one aromatic ring (e.g., benzene) or a polycyclic group having more than one aromatic ring (e.g., two, three, etc. rings). Monocyclic aryl groups may be unsubstituted or substituted as described above with respect to alkyl groups. Regarding polycyclic groups, neighboring aromatic rings may be fused or unfused. The aromatic rings of a polycyclic group may be unsubstituted or substituted as described above with respect to monocyclic aryl groups.

In Formula I and its related formulas, “silyl group” refers to —SiR₃, wherein R is independently selected from hydrogen, alkyl groups, and aryl groups (each of which has been defined herein) and “-” denotes the covalent bond, e.g., to X in Formula I and all its related formulas.

Regarding substituents in the groups of described herein (as opposed to unsubstituted groups), non-hydrogen and non-carbon atoms include, e.g., halogen; oxygen; sulfur; nitrogen; phosphorus; and silicon.

In embodiments, the alkali metal catalysts have Formula I, wherein M is selected from Li, Na, K, Cs and a mixture thereof; X is selected from N and H; R is independently selected from hydrogen, alkyl, and silyl; and n is from 0 to 2, e.g., 0, 1, 2. In embodiments the silyl group is Si(CH₃)₃. In embodiments, the alkyl group is an unsubstituted linear alkyl group. In embodiments, the alkyl group is a substituted cycloalkyl group.

In embodiments, the alkali metal catalysts have Formula IA, MNRₙ, wherein M is selected from Li, Na, K, Cs and a mixture thereof; R is independently selected from hydrogen, alkyl, and silyl; and n is from 1 to 2, e.g., 1, 2. In embodiments the silyl group is Si(CH₃)₃. In embodiments, the alkyl group is an unsubstituted linear alkyl group. In embodiments, the alkyl group is a substituted cycloalkyl group.

In embodiments, the alkali metal catalysts have Formula IB, MNH₂, wherein M is selected from Li, Na, K, Cs and a mixture thereof. In some such embodiments, M is Na. Illustrative alkali metal catalysts according to Formula IB are provided in entries 9-14, 16-23, 26, and 27 of Table 1.

In embodiments, the alkali metal catalysts have Formula IC, MNH, wherein M is selected from Li, Na, K, Cs and a mixture thereof. In some such embodiments, M is Na. Illustrative such alkali metal catalysts are provided in entries 15 and 25 of Table 1.

In embodiments, the alkali metal catalysts have Formula ID, wherein M is selected from Li, Na, K, Cs and a mixture thereof; R is a substituted cycloalkyl group; and n 1. In some such embodiments, M is Na. Illustrative such alkali metal catalysts are provided in entries 33-38 of Table 1.

As noted above, illustrative alkali metal catalysts are listed in Table 1, particularly, entries 6-30 and 33-38.

A single type of alkali metal catalyst or multiple, different types of alkali metal catalysts may be used. The alkali metal catalyst being used to catalyze depolymerization may comprise or consist of any of the disclosed alkali metal catalysts or a combination thereof.

The conditions being used in the present methods may refer to parameters such as the temperature, period of time, atmosphere, and alkali metal catalyst loading. As the present methods may be carried out using a variety types of reactor systems, including batch reactor systems, semi-batch reactor systems, continuous flow reactor systems, and extruder reactor systems (e.g., twin screw extruder reactor system). the conditions may also refer to a specific type of reactor system. These parameters may be adjusted in order to promote depolymerization and thus, to achieve a desired (e.g., maximum) yield of monomer (or other depolymerization product).

Regarding temperature, the temperature may be within 30° C., within 20° C., or within 10° C. of the melting temperature of the polyamide. The temperature may be at or above the melting temperature of the polyamide such that it is in its molten (liquid state) during the depolymerization. In embodiments, the temperature is no more than 300° C., no more than 280° C., no more than 260° C., or no more than 240° C. This includes a range between any of these values and from 200° C. to 280° C., from 200° C. to 250° C., and from 200° C. to 245° C.

Regarding time, this may refer to a total period of time over which the polyamide and the alkali metal catalyst are subjected to depolymerization. In embodiments, the period of time is less than 24 hours, less than 10 hours, less than 5 hours, or less than 2 hours. This includes a range of from 1 minute to 10 hours and 10 minutes to 5 hours. For continuous flow reactor systems, flow rate, rather than time is a relevant parameter. In embodiments, the flow rate is from 5 sccm to 1500 sccm, although higher flow rates may be used. Reactor volume is another relevant parameter for continuous flow reactor systems. In embodiments, the reactor volume is from 50 mL to 1000 L, although greater reactor volumes may be used.

Regarding the atmosphere, a vacuum may be used, e.g., a pressure of 10⁻³ Torr or less, 10⁻² Torr or less, 10⁻¹ Torr or less. However, the method may be carried out at higher pressures, including in a range of from 10⁻² Torr to 10³ Torr. The method may be carried out under an inert atmosphere (e.g., N₂, dry air, argon) and a pressure of about 1 atm (760 Torr).

Regarding alkali metal catalyst loading, the loading may be no more than 20 mol %, no more than 10 mol %, no more than 8 mol %, or no more than 6 mol %. This includes a range of from 0.1 mol % to 5 mol % and from 0.1 mol % to 2 mol %. Mol % is calculated based on the of repeating unit of the polyamide. For example, if 1 g of Nylon-6 is used, the number of mol of repeating unit=0.00885 mol (repeating unit of Nylon-6 is 113 g/mol). Thus, the amount of catalyst used is 0.00885 mol*x mol %.

The present methods are generally carried out without using any liquid medium, e.g., solvent. This includes the methods being carried out without using water or steam. As such, the method may be referred to as being “solvent-free” and the like.

The polyamide (the specific chemical type of which has been described above) may be “virgin” polyamide, which generally refers to pure, as-synthesized polyamide that has not been further processed for use in a particular application (whether the virgin polyamide has been used or not). Alternatively, the polyamide may be “post-consumer” polyamide, which generally refers to a polyamide derived from a consumer product (whether that consumer product has actually been used or not). Post-consumer polyamide may include other components (e.g., other non-polyamide polymers such as polyolefins) such that it may be considered to be a composite with such other components and may have been processed for use in a particular application (e.g., fishing nets, carpet fibers, clothing, medical gloves). In either embodiment, the morphology of the polyamide is not particularly limited. For example, virgin polyamide may be in the form of a powder, including powder composed of micron-sized particles. However, the present methods are capable of achieving high monomer yields from post-consumer polyamide that has been chopped into pieces significantly larger than the particles of virgin polyamide powder. In other words, no milling, e.g., cryogenic milling, is required to achieve high monomer yields from post-consumer polyamide using the present methods. Other morphologies include granules, pellets, films, fibers. Regardless of the source or morphology of the polyamide, prior to use in the methods, the polyamide may be washed and dried by heating under vacuum as described in the Example, below.

When combined for carrying out the present methods, the polyamide and the alkali metal catalyst may be considered to form a reaction mixture comprising each of these components. As noted above, the reaction mixture need not comprise any liquid medium (e.g., solvent). (This does not preclude the presence of a liquid in the reaction mixture due to the use of molten polyamide.) Similarly, the reaction mixture need not comprise other additives. Thus, the reaction mixture may be characterized as being free of a liquid medium (other than the polyamide if in its molten state) and free of an additive. This includes the reaction mixture being free of one or more of the following: an ionic liquid; water or steam; ammonia; N,N-dimethylaminopyridine; acetic anhydride; a phosphoric acid (or salt thereof); a boric acid (or salt thereof); a sulfonic acid (or salt thereof); a carboxylic acid (or salt thereof); a carbonate; an alkali or alkaline earth oxide; an alkali or alkaline earth hydroxide; an alkali or alkaline earth carbonate; an alkali or alkaline earth carboxylate; an alcohol. In embodiments, the reaction mixture consists of the polyamide and the alkali metal catalyst. These embodiments do not preclude the ultimate presence of monomers provided by the depolymerization, unreacted polyamide and/or other depolymerized product (e.g., polyamide fragments) in the reaction mixture. These embodiments also do not preclude the presence of components or impurities which may be inherently present in the reaction mixture due to the particular synthetic technique used to form the polyamide. These embodiments also do not preclude the presence of components which may be inherently present in the reaction mixture due to the source of the polyamide (e.g., post-consumer polyamide).

The present methods may be carried out using a variety types of reactor systems, including batch reactor systems, semi-batch reactor systems, and continuous flow reactor systems. As noted throughout, the present methods are capable of achieving continuous operation for a period of time (e.g., by using a continuous flow of polyamide) while maintaining high monomer yields.

The present methods may further comprise recovering any products and/or unreacted material from the reaction mixture. This includes recovering and/or recycling the alkali metal catalysts (or a derivative thereof) from the reaction mixture. No liquid medium is required for recovery of the alkali metal catalysts. The recovered alkali metal catalysts may be used to carry out the method one or more additional times (i.e., they may be recycled). Similarly, the present methods may further comprise recovering the depolymerized product, e.g., monomers, and using them for any desired purpose, including synthesizing a new polymer, including a new polyamide. Recovery of the depolymerized products may be by vaporization or sublimation.

The present methods may be characterized by a yield of monomer. The yield is reported as (weight of monomer)/(starting weight of polyamide)*100%. The yield may be determined using 1H NMR as described in the Example below. The yield may be an initial yield obtained by using fresh (i.e., unused) alkali metal catalyst. The initial yield may be at least 70%, at least 75%, at least 80%, at least 85%, at least 90%, at least 95%, or at least 98%. This includes a range between any of these values, as well as from 70% to 100%, from 80% to 100%, and from 80% to 95%. The yield may be a yield obtained by using recovered alkali metal catalyst which has been used one or more times (e.g., 1, 2, 3, 4, etc.). The yield from a recycled/recovered alkali metal catalyst may be within =50%, +20%, +10%, +5%, or +2% of the initial yield. Any of the yield values in this paragraph may refer to a specific polyamide (e.g., Nylon-6), a specific monomer (e.g., ε-caprolactam), a specific alkali metal catalyst (e.g., any of those in entries 6-30 and 33-38 of Table 1) and the method carried out at under specific conditions (e.g., any of those in Tables 1-3).

As noted above, the polyamide to be depolymerized by the present methods may be provided in a blend with non-polyamide polymers, e.g., a polyolefin. Other non-polyamide polymers include those used in the Example, below. The method may depolymerize the polyamide while leaving the other polymer intact. The term “intact” does not necessarily mean perfectly intact as a minor amount of the non-polyamide polymer may be decomposed. Thus, the present methods allow for separation of a polyamide from such a blend. The Example below also demonstrates that the present methods may be used to selectively depolymerize a certain type of polyamide (e.g., Nylon-6) in a blend with other different types of polyamides (e.g., Nylon 12, Nylon 66), enabling separation of the different types of polyamides from one another.

The present disclosure encompasses any of the alkali metal catalysts described herein as well as reaction mixtures comprising (or consisting of) such catalysts with any of the disclosed polyamides.

### EXAMPLE

### Introduction

This application teaches a novel approach to efficiently depolymerize Nylon-6 to ε-caprolactam using Earth-abundant, low-cost, metal-organic and inorganic alkali metal superbases with quantitative conversions, short reaction times (e.g., as low as 10 minutes), low temperatures (e.g., as low as 220° C.), and low catalyst loadings (e.g., as low as 0.2 wt %). These catalysts function equally well under a vacuum or inert atmosphere. In this Example, the performance of these “super basic” catalysts to other catalysts that have been used in existing techniques, including K₂CO₃, Na₂CO₃, and KOH, under identical reaction conditions. The results clearly demonstrate that the present super basic catalysts exhibit significantly higher conversion rates and require lower reaction temperatures than catalysts that have been used in existing techniques. Additionally, the superbases can be used in continuous operation without reducing the reaction rate. The resulting ε-caprolactam can be polymerized back to pristine Nylon-6, demonstrating that this approach is an alternative to current methods for Nylon-6 depolymerization. The findings indicate that the superbase catalysts offer an efficient and sustainable technology for Nylon-6 recycling for a wide range of industrial applications.

### Experimental

### General Methods and Materials

Methods: All depolymerization reactions were assembled by mixing the polymer and the appropriate catalyst in a N₂-filled MBraun glovebox in cylindrical 50 mL Schlenk tubes. Heating is supplied by a customized aluminum heating block with a fitted hole for the Schlenk tubes.

General materials: All alkali metal catalysts were purchased from commercial sources, and used directly without further purification. Pristine Nylon-6 powder with a mean particle size of 15-20 μm and a molecular weight of 11930 g/mol (as determined by GPC) was obtained from Goodfellow Inc. The Nylon-6 powder was washed with 1 M KOH solution overnight, filtered and washed with H₂O, and dried under a high vacuum at 100° C. for at least 24 h prior to use. Nylon-6 Pellets were purchased from Sigma Aldrich and used as received. The Fishing net was purchased from Amazon.

### Physical and Analytical Methods

NMR spectra were recorded on a Varian Bruker Avance III HD system equipped with a TXO Prodigy probe (500 MHz) spectrometer. Chemical shifts (8) for ¹H-NMR are referenced to the internal solvent. Gel permeation chromatography (GPC) was used to analyze molecular weight and molecular weight dispersity indices of synthesized Nylon-6.

### General Depolymerization Procedure

For some experiments, the following procedure (1) was used. In a glove box, a 50 mL oven-dried Schlenk tube was charged with a magnetic stir bar, Nylon-6 polymer, and finely ground catalyst. The vessel was sealed tightly, and the polymer and catalyst were thoroughly mixed by stirring at room temperature for approximately 5 min. The Schlenk tube was then evacuated to 10⁻³ Torr, sealed, and heated to the specified temperature with slow magnetic stirring (50-100 rpm) for the specified time. Reaction time was recorded starting 2.5 min after the reaction tube was placed in the heating block, since it takes an average of 2.5 min for the reaction to reach the depolymerization temperature and the polymer to begin melting. During the reaction, the products sublime from the hot reaction zone and deposit as a crystalline layer on the cold wall of the reactor. After cooling to room temperature, the soluble part of the reaction mixture was dissolved in 3-4 mL of deuterated solvent, and mesitylene was added as an internal standard. A sample of this solution was withdrawn for NMR analysis. Yields were determined by ¹H-NMR, comparing the signal integrals of ε-caprolactam and mesitylene. For other experiments, the following procedure (2) was used. Air-stored Nylon-6 was loaded into a three-neck round bottom flask equipped with an overhead stirrer and a cold trap. Flask sizes ranged from 3 to 12 liters. Under reduced pressure (100-500 mTorr), the flask was heated until all the Nylon-6 melted. Once melted, the reactor was purged with argon gas, and the catalyst was introduced. The catalyst can also be introduced before the Nylon-6 melts. The reactor was sealed, and a vacuum was applied to the reaction mixture. Rapid bubbling occurred in the reaction flask, indicating product formation and evaporation, while simultaneously, the product was collected in the cold trap. Upon completion of the reaction, the cold trap was allowed to return to room temperature, and the product was physically removed from the cold trap. Yields were determined by weighing the collected products, and the purity of caprolactam was assessed using NMR spectroscopy. To further enhance the purity of the caprolactam, additional vacuum distillation of the contents of the cold trap may be conducted at a temperature range of 120-150° C.

### Procedures and NMR Spectra for Reactions in Table 1

### Table 1, Entry 1

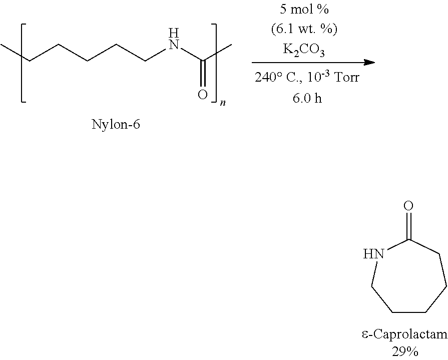

Exactly 100 mg of Nylon-6 powder (0.89 mmol) and 6.1 mg of K₂CO₃ (5 mol %, 6.1 wt. %) were reacted according to the general procedure, heating the reaction mixture to 240° C. for 6.0 h. Exactly 18.5 mg of mesitylene (0.154 mmol) was used as an internal standard. ε-caprolactam was obtained in 29% yield.

### Table 1, Entry 2

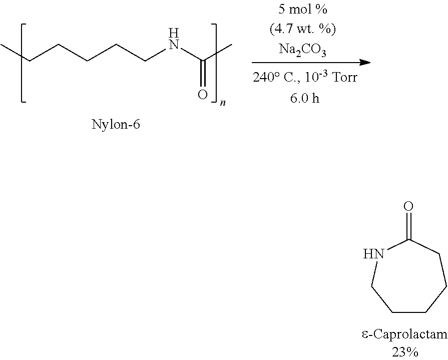

Exactly 100 mg of Nylon-6 powder (0.89 mmol) and 4.7 mg of Na₂CO₃ (5 mol %, 4.7 wt. %) were reacted according to the general procedure, heating the reaction mixture to 240° C. for 6.0 h. Exactly 22.9 mg of mesitylene (0.191 mmol) was used as an internal standard. ε-caprolactam was obtained in 23% yield.

### Table 1, Entry 3

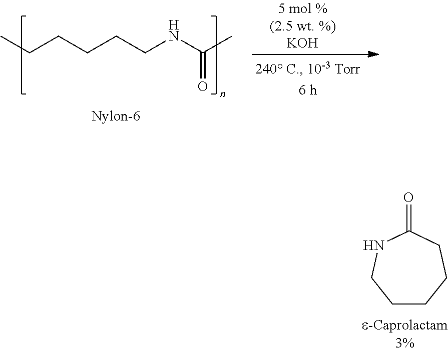

Exactly 100 mg of Nylon-6 powder (0.89 mmol) and 4.7 mg of KOH (5 mol %, 2.5 wt. %) were reacted according to the general procedure, heating the reaction mixture to 240° C. for 6.0 h. Exactly 19.6 mg of mesitylene (0.163 mmol) was used as an internal standard. ε-caprolactam was obtained in 3% yield.

### Table 1, Entry 4

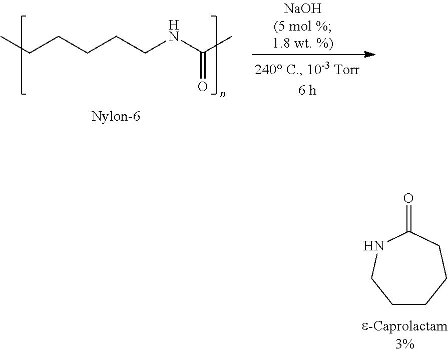

Exactly 100 mg of Nylon-6 powder (0.89 mmol) and 1.8 mg of NaOH (5 mol %, 1.8 wt. %) were reacted according to the general procedure, heating the reaction mixture to 240° C. for 6 h. Exactly 26.3 mg of mesitylene (0.218 mmol) was used as an internal standard. ε-caprolactam was obtained in 3% yield.

### Table 1, Entry 5

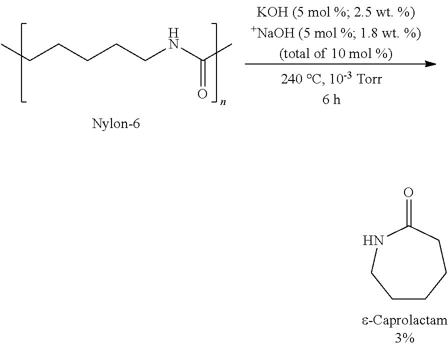

Exactly 100 mg of Nylon-6 powder (0.89 mmol), 1.8 mg of NaOH (5 mol %, 1.8 wt. %) and 2.5 mg of KOH (5 mol %, 2.5 wt. %) were reacted according to the general procedure, heating the reaction mixture to 240° C. for 6 h. Exactly 22.8 mg of mesitylene (0.190 mmol) was used as an internal standard. ε-caprolactam was obtained in 3% yield.

### Table 1, Entry 6

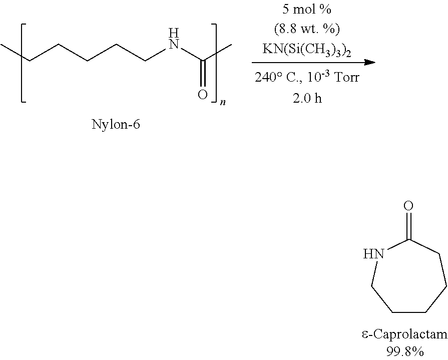

Exactly 99.8 mg of Nylon-6 powder (0.89 mmol) and 8.8 mg of KN(Si(CH₃)₃) 2 (5 mol %, 8.8 wt. %) were reacted according to the general procedure, heating the reaction mixture to 240° C. for 2.0 h. Exactly 23.2 mg of mesitylene (0.193 mmol) was used as an internal standard. ε-caprolactam was obtained in 99.8% yield.

### Table 1, Entry 7

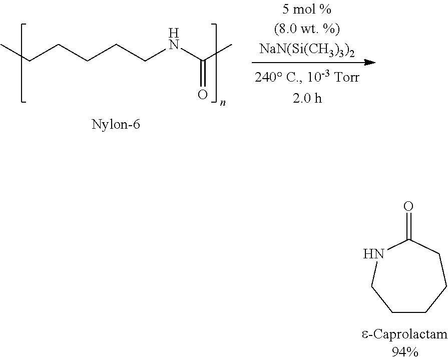

Exactly 100 mg of Nylon-6 powder (0.89 mmol) and 8.0 mg of NaN(Si(CH₃)₃) 2 (5 mol %, 8.0 wt. %) were reacted according to the general procedure, heating the reaction mixture to 240° C. for 2.0 h. Exactly 22.2 mg of mesitylene (0.185 mmol) was used as an internal standard. ε-caprolactam was obtained in 94% yield.

### Table 1, Entry 8

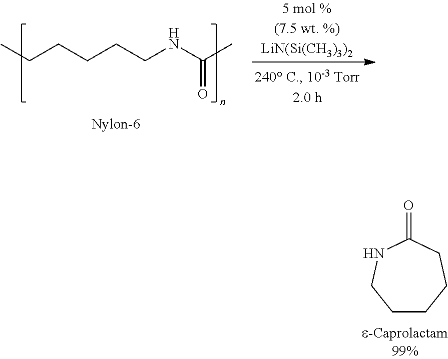

Exactly 100 mg of Nylon-6 powder (0.89 mmol) and 7.5 mg of LiN(Si(CH₃)₃) 2 (5 mol %, 7.5 wt. %) were reacted according to the general procedure, heating the reaction mixture to 240° C. for 2.0 h. Exactly 31.8 mg of mesitylene (0.265 mmol) was used as an internal standard. ε-caprolactam was obtained in 99% yield.

### Table 1, Entry 9

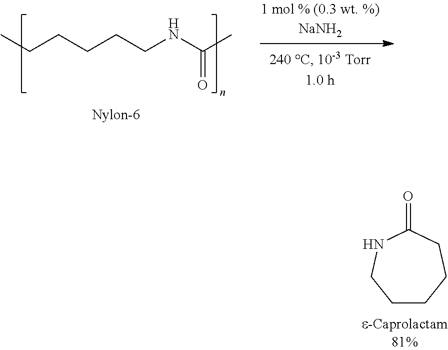

Exactly 100 mg of Nylon-6 powder (0.89 mmol) and 0.3 mg of NaNH₂ (1 mol %, 0.3 wt. %) were reacted according to the general procedure, heating the reaction mixture to 240° C. for 1.0 h. Exactly 24.0 mg of mesitylene (0.200 mmol) was used as an internal standard. ε-caprolactam was obtained in 81% yield.

### Table 1, Entry 10

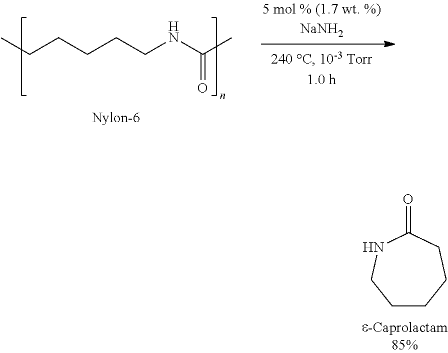

Exactly 100 mg of Nylon-6 powder (0.89 mmol) and 1.7 mg of NaNH₂ (5 mol %, 1.7 wt. %) were reacted according to the general procedure, heating the reaction mixture to 240° C. for 1.0 h. Exactly 22.5 mg of mesitylene (0.187 mmol) was used as an internal standard. ε-caprolactam was obtained in 85% yield.

### Table 1, Entry 11

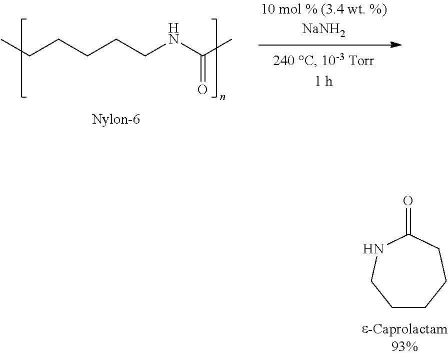

Exactly 100 mg of Nylon-6 powder (0.89 mmol) and 3.4 mg of NaNH₂ (10 mol %, 3.4 wt. %) were reacted according to the general procedure, heating the reaction mixture to 240° C. for 1 h. Exactly 19.1 mg of mesitylene (0.159 mmol) was used as an internal standard. ε-caprolactam was obtained in 93% yield.

### Table 1, Entry 12

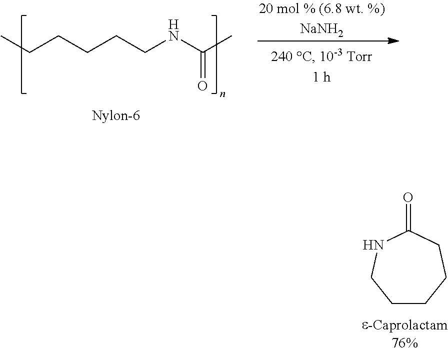

Exactly 100 mg of Nylon-6 powder (0.89 mmol) and 6.8 mg of NaNH₂ (20 mol %, 6.8 wt. %) were reacted according to the general procedure, heating the reaction mixture to 240° C. for 1 h. Exactly 29.0 mg of mesitylene (0.241 mmol) was used as an internal standard. ε-caprolactam was obtained in 76% yield.

### Table 1, Entry 13

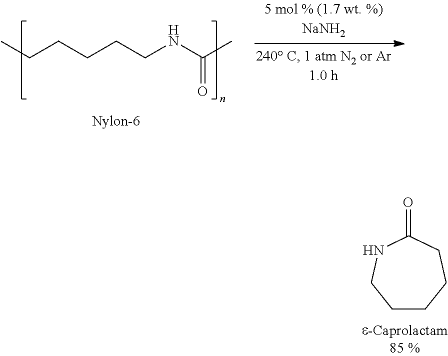

Exactly 100 mg of Nylon-6 powder (0.89 mmol) and 1.7 mg of NaNH₂ (5 mol %, 1.7 wt. %) were reacted according to the general procedure without applying static vacuum, heating the reaction mixture to 240° C. for 1.0 h. Exactly 23.2 mg of mesitylene (0.193 mmol) was used as an internal standard. ε-caprolactam was obtained in 85% yield.

### Table 1, Entry 14

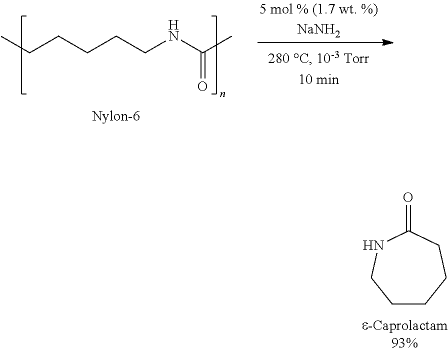

Exactly 100 mg of Nylon-6 powder (0.89 mmol) and 1.7 mg of NaNH₂ (5 mol %, 1.7 wt. %) were reacted according to the general procedure, heating the reaction mixture to 280° C. for 10 min. Exactly 20.0 mg of mesitylene (0.166 mmol) was used as an internal standard. ε-caprolactam was obtained in 93% yield.

### Table 1, Entry 15

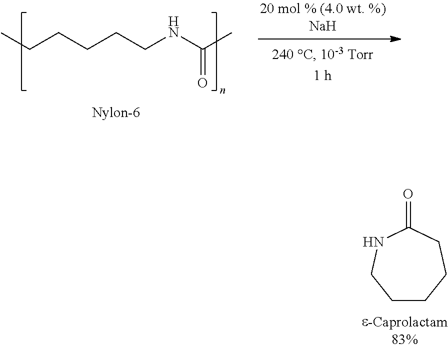

Exactly 100 mg of Nylon-6 powder (0.89 mmol) and 4.0 mg of NaH (20 mol %, 4.0 wt. %) were reacted according to the general procedure, heating the reaction mixture to 240° C. for 1 h. Exactly 21.5 mg of mesitylene (0.179 mmol) was used as an internal standard. ε-caprolactam was obtained in 83% yield.

### Table 1, Entry 16

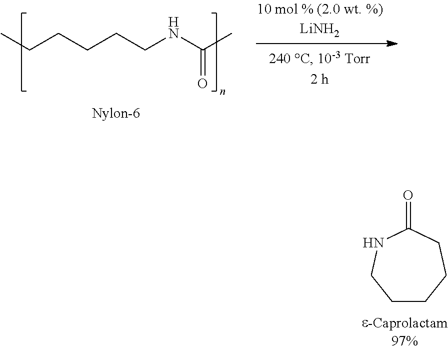

Exactly 100 mg of Nylon-6 powder (0.89 mmol) and 2.0 mg of LiNH₂ (10 mol %, 2.0 wt. %) were reacted according to the general procedure, heating the reaction mixture to 240° C. for 2 h. Exactly 26.6 mg of mesitylene (0.221 mmol) was used as an internal standard. ε-caprolactam was obtained in 97% yield.

### Table 1, Entry 17

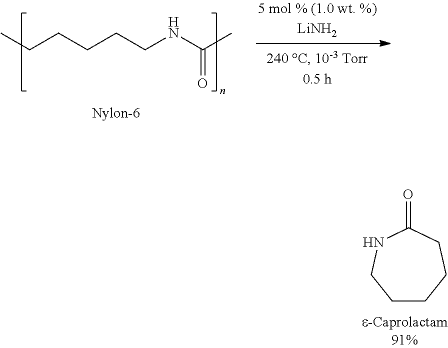

Exactly 100 mg of Nylon-6 powder (0.89 mmol) and 1.0 mg of LiNH₂ (5 mol %, 1.0 wt. %) were reacted according to the general procedure, heating the reaction mixture to 240° C. for 0.5 h. Exactly 21.5 mg of mesitylene (0.179 mmol) was used as an internal standard. ε-caprolactam was obtained in 91% yield.

### Table 1, Entry 18

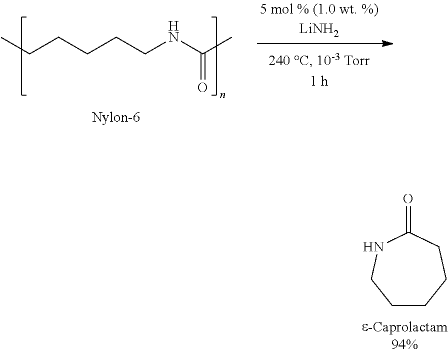

Exactly 100 mg of Nylon-6 powder (0.89 mmol) and 1.0 mg of LiNH₂ (5 mol %, 1.0 wt. %) were reacted according to the general procedure, heating the reaction mixture to 240° C. for 1 h. Exactly 28.0 mg of mesitylene (0.233 mmol) was used as an internal standard. ε-caprolactam was obtained in 94% yield.

### Table 1, Entry 19

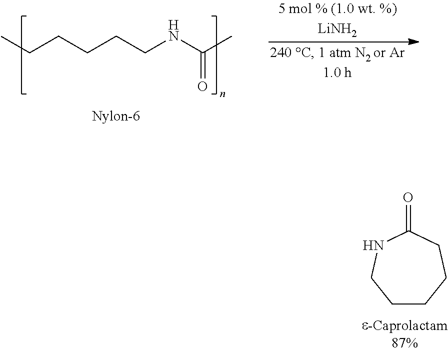

Exactly 100 mg of Nylon-6 powder (0.89 mmol) and 1.0 mg of LiNH₂ (5 mol %, 1.0 wt. %) were reacted according to the general procedure without applying static vacuum, heating the reaction mixture to 240° C. for 1 h. Exactly 22.7 mg of mesitylene (0.189 mmol) was used as an internal standard. ε-caprolactam was obtained in 87% yield.

### Table 1, Entry 20

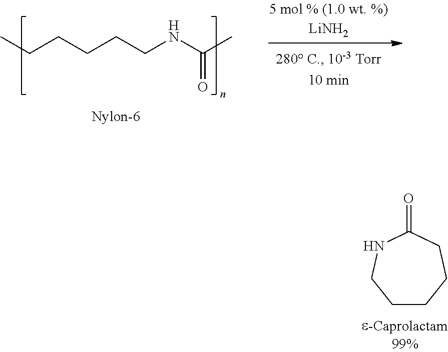

Exactly 100 mg of Nylon-6 powder (0.89 mmol) and 1.0 mg of LiNH₂ (5 mol %, 1.0 wt. %) were reacted according to the general procedure, heating the reaction mixture to 280° C. for 10 min. Exactly 16.7 mg of mesitylene (0.139 mmol) was used as an internal standard. ε-caprolactam was obtained in 99% yield.

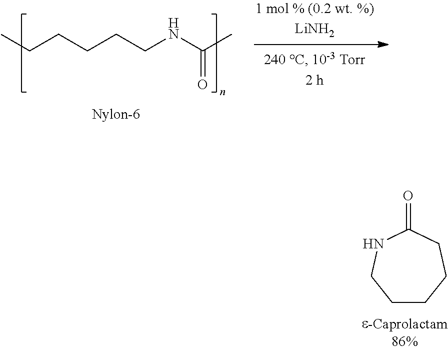

### Table 1, Entry 21

Exactly 100 mg of Nylon-6 powder (0.89 mmol) and 0.2 mg of LiNH₂ (1 mol %, 0.2 wt. %) were reacted according to the general procedure, heating the reaction mixture to 240° C. for 2 h. Exactly 25.3 mg of mesitylene (0.211 mmol) was used as an internal standard. ε-caprolactam was obtained in 86% yield.

### Table 1, Entry 22

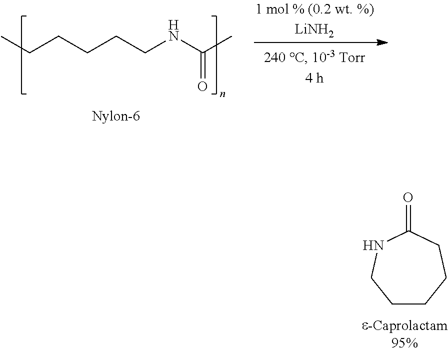

Exactly 100 mg of Nylon-6 powder (0.89 mmol) and 0.2 mg of LiNH₂ (1 mol %, 0.2 wt. %) were reacted according to the general procedure, heating the reaction mixture to 240° C. for 4 h. Exactly 22.2 mg of mesitylene (0.185 mmol) was used as an internal standard. ε-caprolactam was obtained in 95% yield.

### Table 1, Entry 23

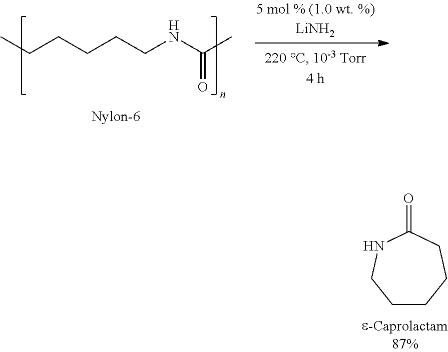

Exactly 100 mg of Nylon-6 powder (0.89 mmol) and 1.0 mg of LiNH₂ (5 mol %, 1.0 wt. %) were reacted according to the general procedure, heating the reaction mixture to 220° C. for 4 h. Exactly 23.8 mg of mesitylene (0.198 mmol) was used as an internal standard. ε-caprolactam was obtained in 87% yield.

### Table 1, Entry 24

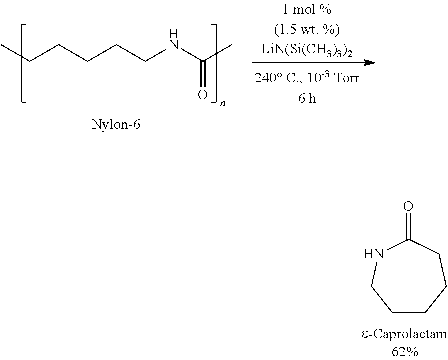

Exactly 100 mg of Nylon-6 powder (0.89 mmol) and 1.5 mg of LiN(Si(CH₃)₃) 2 (1 mol %, 1.5 wt. %) were reacted according to the general procedure, heating the reaction mixture to 240° C. for 6 h. Exactly 35.2 mg of mesitylene (0.293 mmol) was used as an internal standard. ε-caprolactam was obtained in 62% yield.

### Table 1, Entry 25

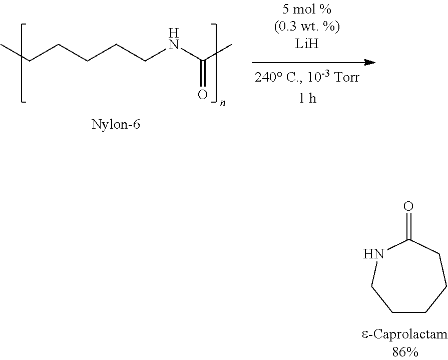

Exactly 100 mg of Nylon-6 powder (0.89 mmol) and 0.3 mg of LiH (5 mol %, 0.3 wt. %) were reacted according to the general procedure, heating the reaction mixture to 240° C. for 1 h. Exactly 23.5 mg of mesitylene (0.196 mmol) was used as an internal standard. ε-caprolactam was obtained in 86% yield.

### Table 1, Entry 26

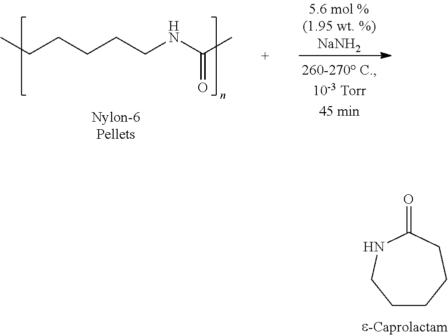

To 100 gr of molten Nylon-6 pellets with a moisture content of ~1%, 1.95 gr of NaNH₂ (5.65 mol %, 1.95 wt. %) was added under Ar flow. The reactor was sealed, and a vacuum was applied to the reaction mixture. The reaction mixture was heated for 45 min in the temperature range of (260-270° C.) during which 86 gr of the product was collected in the cold trap. The ε-caprolactam purity was 95.6%.

### Table 1, Entry 27

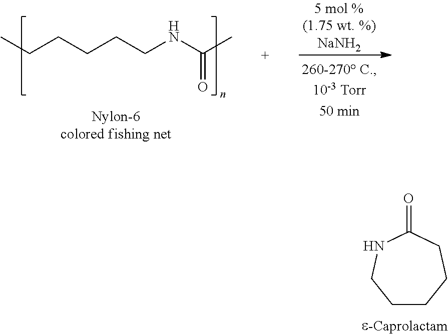

To 100 gr of molten Nylon-6 colored fishing net with a moisture content of 1.75%, 1.75 gr of NaNH₂ (5 mol %, 1.75 wt. %) was added under Ar flow. The reactor was sealed, and a vacuum was applied to the reaction mixture. The reaction mixture was heated for 50 min in the temperature range of (240-250° C.) during which 90.57 gr of the product was collected in the cold trap. The ε-caprolactam purity was 95.2%. The collected fraction was subjected to vacuum distillation at 10⁻¹ Torr at 140° C., leaving behind 4.6% of a caprolactam dimer. The collected white ε-caprolactam had a purity of 99%.

### Table 1, Entry 28

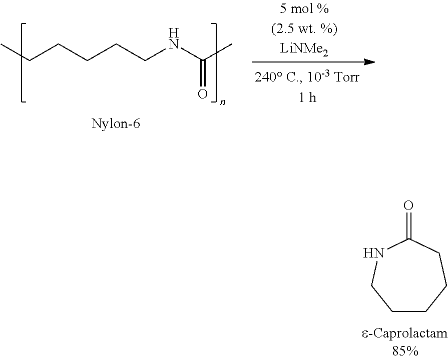

Exactly 100 mg of Nylon-6 powder (0.89 mmol) and 2.5 mg of LiNMe₂ (5 mol %, 2.5 wt. %) were reacted according to the general procedure, heating the reaction mixture to 240° C. for 1 h. Exactly 22.0 mg of mesitylene (0.183 mmol) was used as an internal standard. ε-caprolactam was obtained in 85% yield.

### Table 1, Entry 29

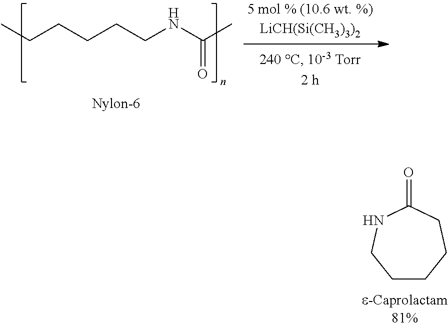

Exactly 100 mg of Nylon-6 powder (0.89 mmol) and 10.6 mg of LiCH(Si(CH₃)₃) 2 (5 mol %, 10.6 wt. %) were reacted according to the general procedure, heating the reaction mixture to 240° C. for 2 h. Exactly 19.1 mg of mesitylene (0.159 mmol) was used as an internal standard. ε-caprolactam was obtained in 81% yield.

### Table 1, Entry 30

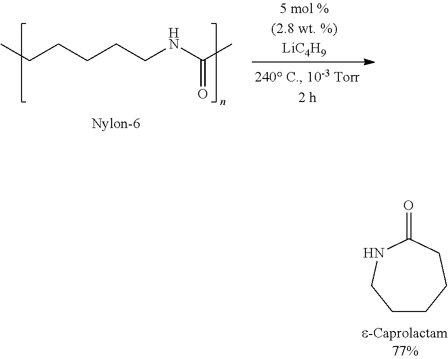

Exactly 0.5 mL of nBuLi solution (in hexane; 1.6 M) was diluted with dry pentane (17.6 mL) to prepare a stock solution with a concentration of 0.0442 M. Exactly 100 mg of Nylon-6 powder (0.89 mmol) and 1 mL of the above stock solution of nBuLi (5 mol %, 0.0442 mmol) were mixed, followed by the removal of hexane and pentane under reduced pressure for 3 hours. According to the general procedure, the mixture was heated to 240° C. for 2 h. Exactly 22.0 mg of mesitylene (0.183 mmol) was used as an internal standard. ε-caprolactam was obtained in 77% yield.

### Table 1, Entry 31

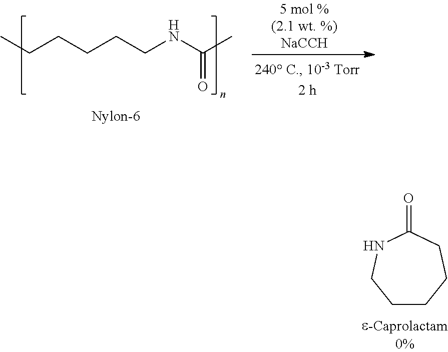

11.7 mg of Sodium acetylide suspension (18 wt. % slurry in xylene: light mineral oil; Sigma-Aldrich) was added to exactly 100 mg of Nylon-6 powder (0.89 mmol), followed by the removal of solvents under reduced pressure for 72 hours. According to the general procedure, the mixture was heated to 240° C. for 2 h. No caprolactam was obtained.

### Table 1, Entry 32

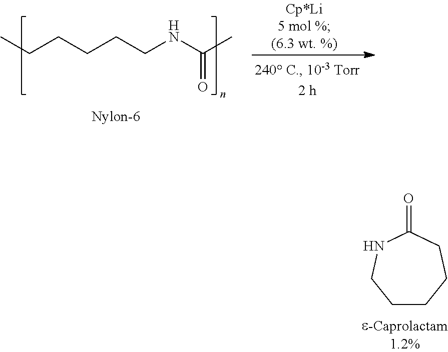

Exactly 100 mg of Nylon-6 powder (0.89 mmol) and 6.3 mg of Cp*Li (5 mol %, 6.3 wt. %) were reacted according to the general procedure, heating the reaction mixture to 240° C. for 2 h. Exactly 26.3 mg of mesitylene (0.185 mmol) was used as an internal standard. ε-caprolactam was obtained in 1.2% yield.

### Table 1, Entry 33

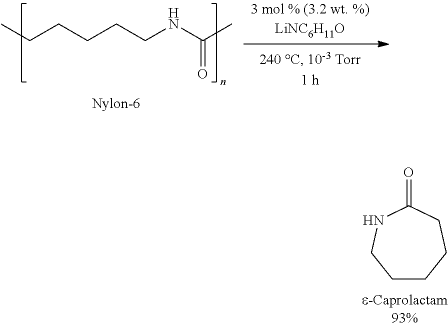

Exactly 100 mg of Nylon-6 powder (0.89 mmol) 3.2 mg of LiNC₆H₁₁O (3 mol %, 3.2 wt. %) were reacted according to the general procedure, heating the reaction mixture to 240° C. for 1 h. Exactly 17.0 mg of mesitylene (0.141 mmol) was used as an internal standard. ε-caprolactam was obtained in 93% yield.

### Table 1, Entry 34

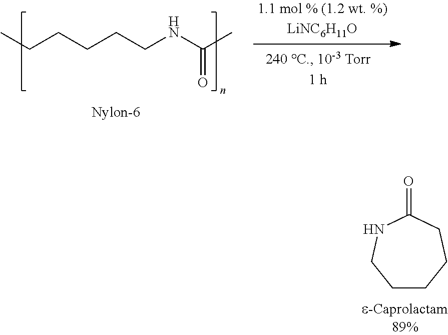

Exactly 100 mg of Nylon-6 powder (0.89 mmol) 1.2 mg of LiNC₆H₁₁O (1.1 mol %, 1.2 wt. %) were reacted according to the general procedure, heating the reaction mixture to 240° C. for 1 h. Exactly 23.7.0 mg of mesitylene (0.197 mmol) was used as an internal standard. ε-caprolactam was obtained in 89% yield.

### Table 1, Entry 35

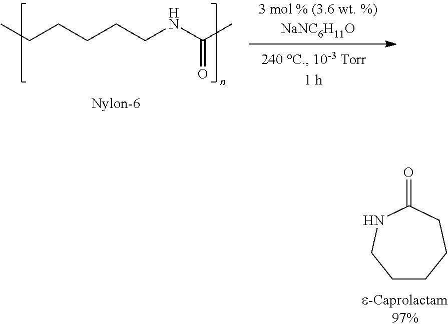

Exactly 100 mg of Nylon-6 powder (0.89 mmol) and 3.6 mg of NaNC₆H₁₁O (3 mol %, 3.6 wt. %) were reacted according to the general procedure, heating the reaction mixture to 240° C. for 1 h. Exactly 19.0 mg of mesitylene (0.158 mmol) was used as an internal standard. ε-caprolactam was obtained in 97% yield.

### Table 1, Entry 36

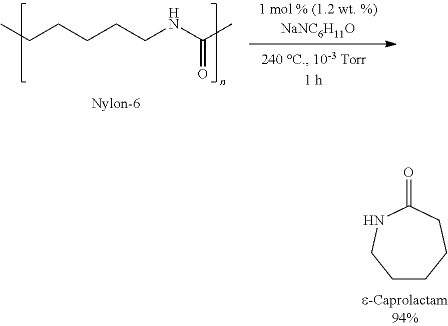

Exactly 100 mg of Nylon-6 powder (0.89 mmol), and 1.2 mg of NaNC₆H₁₁O (1 mol %, 1.2 wt. %) were reacted according to the general procedure, heating the reaction mixture to 240° C. for 1 h. Exactly 17.3 mg of mesitylene (0.143 mmol) was used as an internal standard. ε-caprolactam was obtained in 94% yield.

### Table 1, Entry 37

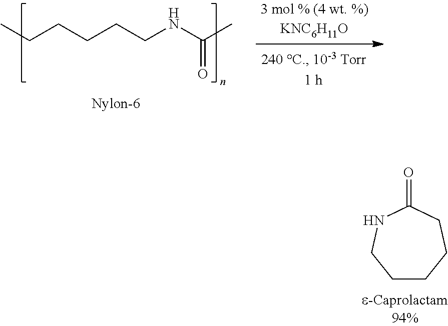

Exactly 100 mg of Nylon-6 powder (0.89 mmol) and 4.0 mg of KNC₆H₁₁O (3 mol %, 4 wt. %) were reacted according to the general procedure, heating the reaction mixture to 240° C. for 1 h. Exactly 25.5 mg of mesitylene (0.212 mmol) was used as an internal standard. ε-caprolactam was obtained in 94% yield.

### Table 1, Entry 38

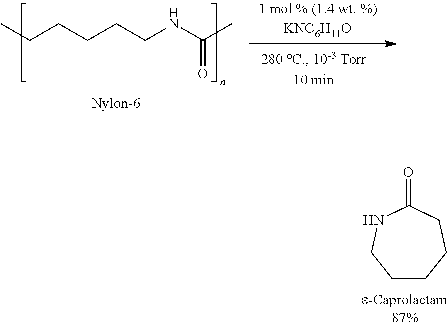

Exactly 100 mg of Nylon-6 powder (0.89 mmol) and 1.4 mg of KNC₆H₁₁O (3 mol %, 4 wt. %) were reacted according to the general procedure, heating the reaction mixture to 240° C. for 1 h. Exactly 17.5 mg of mesitylene (0.145 mmol) was used as an internal standard. ε-caprolactam was obtained in 87% yield.

### Table 2, Entry 1

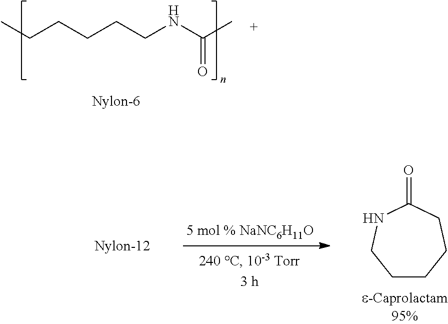

Exactly 60 mg of Nylon-6 powder (0.45 mmol), 50 mg of Nylon-12 powder and 3.6 mg of NaNC₆H₁₁O (5 mol %, 6 wt. %) were reacted according to the general procedure, heating the reaction mixture to 240° C. for 3 h. Exactly 18.0 mg of mesitylene (0.149 mmol) was used as an internal standard. ε-caprolactam was obtained in 95% yield.

### Table 2, Entry 2

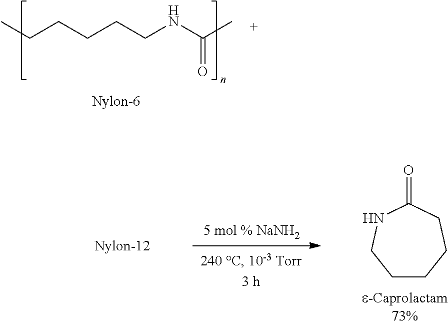

Exactly 50 mg of Nylon-6 powder (0.45 mmol), 50 mg of Nylon-12 powder and 0.87 mg of NaNH₂ (5 mol %, 1.74 wt. %) were reacted according to the general procedure, heating the reaction mixture to 240° C. for 3 h. Exactly 25.0 mg of mesitylene (0.208 mmol) was used as an internal standard. ε-caprolactam was obtained in 73% yield.

### Table 2, Entry 3

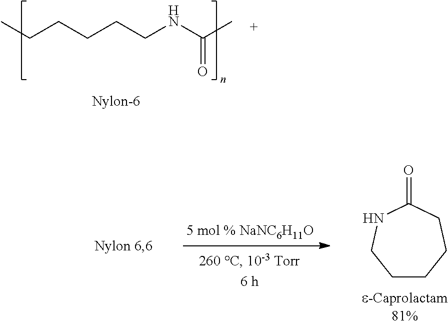

Exactly 50 mg of Nylon-6 powder (0.45 mmol), 50 mg of Nylon-6,6 powder and 3 mg of NaNC₆H₁₁O (5 mol %, 6 wt. %) were reacted according to the general procedure, heating the reaction mixture to 260° C. for 6 h. Exactly 24.0 mg of mesitylene (0.199 mmol) was used as an internal standard. ε-caprolactam was obtained in 81% yield.

### Table 2, Entry 4

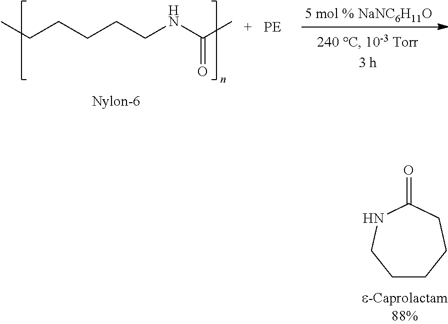

Exactly 50 mg of Nylon-6 powder (0.45 mmol), 50 mg of PE (polyethylene homopolymer) powder and 3 mg of NaNC₆H₁₁O (5 mol %, 6 wt. %) were reacted according to the general procedure, heating the reaction mixture to 240° C. for 3 h. Exactly 19.0 mg of mesitylene (0.158 mmol) was used as an internal standard. ε-caprolactam was obtained in 88% yield.

### Table 2, Entry 5

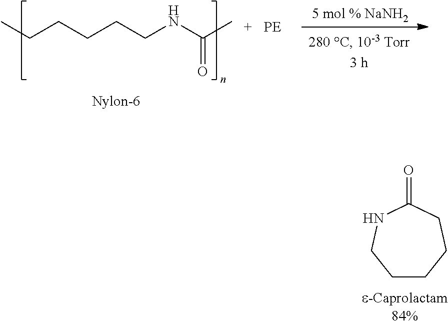

Exactly 50 mg of Nylon-6 powder (0.45 mmol), 16.67 mg of PE (isotactic polypropylene) powder and 0.87 mg of NaNH₂ (5 mol %, 1.74 wt. %) were reacted according to the general procedure, heating the reaction mixture to 280° C. for 3 h. Exactly 19.0 mg of mesitylene (0.158 mmol) was used as an internal standard. ε-caprolactam was obtained in 84% yield.

### Table 2, Entry 6

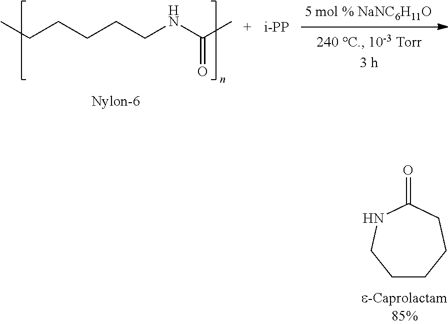

Exactly 50 mg of Nylon-6 powder (0.45 mmol), 50 mg of i-PP powder and 3 mg of NaNC₆H₁₁O (5 mol %, 6 wt. %) were reacted according to the general procedure, heating the reaction mixture to 240° C. for 3 h. Exactly 19.0 mg of mesitylene (0.158 mmol) was used as an internal standard. ε-caprolactam was obtained in 85% yield.

### Table 2, Entry 7

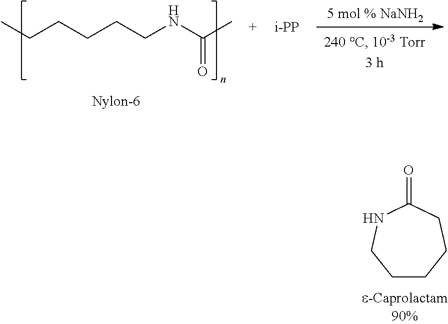

Exactly 50 mg of Nylon-6 powder (0.45 mmol), 50 mg of i-PP powder and 0.87 mg of NaNH₂ (5 mol %, 1.74 wt. %) were reacted according to the general procedure, heating the reaction mixture to 240° C. for 3 h. Exactly 23.0 mg of mesitylene (0.191 mmol) was used as an internal standard. ε-caprolactam was obtained in 90% yield.

### Table 2, Entry 8

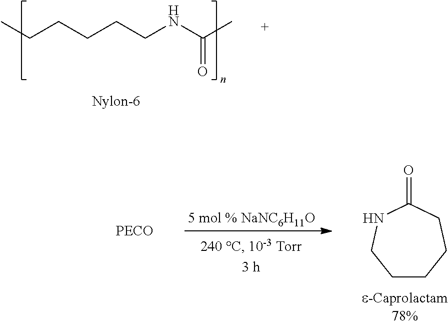

Exactly 50 mg of Nylon-6 powder (0.45 mmol), 50 mg of PECO (polyethylene-co-1-octene) and 3 mg of NaNC₆H₁₁O (5 mol %, 6 wt. %) were reacted according to the general procedure, heating the reaction mixture to 240° C. for 3 h. Exactly 20.0 mg of mesitylene (0.149 mmol) was used as an internal standard. ε-caprolactam was obtained in 78% yield.

### Table 2, Entry 9

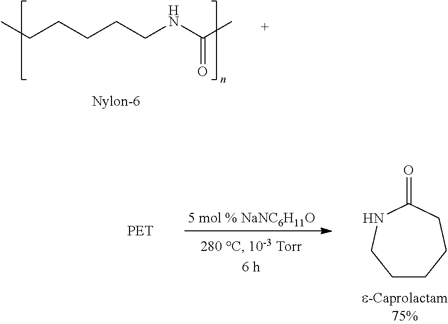

Exactly 71 mg of Nylon-6 powder (0.45 mmol), 30 mg of PET (polyethylene terephthalate) powder and 27 mg of a 1:5.55 NaNC₆H₁₁O: caprolactam mixture (5.7 mol %) were reacted according to the general procedure, heating the reaction mixture to 240° C. for 6 h. Exactly 21.6 mg of mesitylene (0.179 mmol) was used as an internal standard. After subtracting the additional caprolactam from the NaNC₆H₁₁O: caprolactam mixture, ε-caprolactam was obtained in 75% yield.

### Table 2, Entry 10

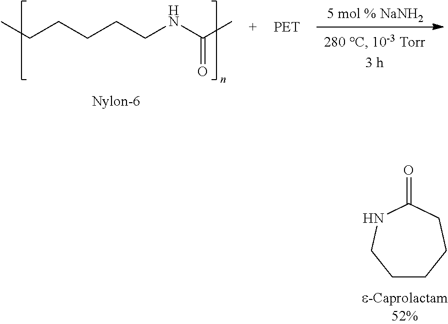

Exactly 50 mg of Nylon-6 powder (0.45 mmol), 5 mg of PET powder and 0.87 mg of NaNH₂ (5 mol %, 1.74 wt. %) were reacted according to the general procedure, heating the reaction mixture to 280° C. for 3 h. Exactly 21.0 mg of mesitylene (0.175 mmol) was used as an internal standard. ε-caprolactam was obtained in 52% yield.

### Table 2, Entry 11

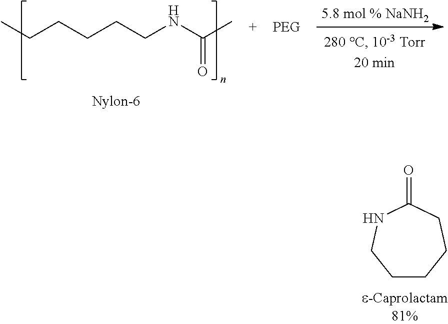

Exactly 100 mg of Nylon-6 powder (0.45 mmol), 20 mg of PEG (polyethyleneglycol) powder and 2 mg of NaNH₂ (5.8 mol %, 2 wt. %) were reacted according to the general procedure, heating the reaction mixture to 280° C. for 20 min. Exactly 19.3 mg of mesitylene (0.160 mmol) was used as an internal standard. ε-caprolactam was obtained in 81% yield.

### Table 2, Entry 12

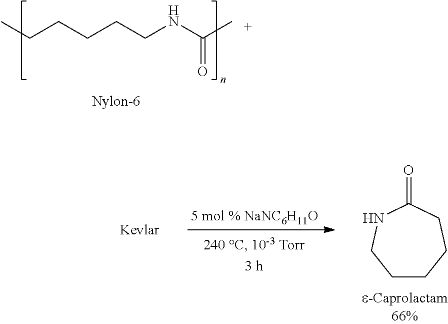

Exactly 50 mg of Nylon-6 powder (0.45 mmol), 50 mg of Kevlar fiber, taken from a Kevlar glove, and 3 mg of NaNC₆H₁₁O (5 mol %, 6 wt. %) were reacted according to the general procedure, heating the reaction mixture to 240° C. for 3 h. Exactly 25.0 mg of mesitylene (0.208 mmol) was used as an internal standard. ε-caprolactam was obtained in 66% yield.

### Table 2, Entry 13

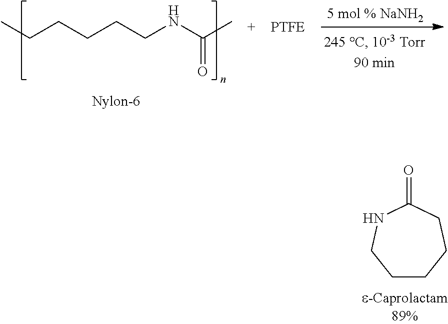

Exactly 100 mg of Nylon-6 powder (0.88 mmol), 33 mg of PTFE strips, taken from a Teflon tape, and 1.73 mg of NaNH₂ (5 mol %, 1.73 wt. %) were reacted according to the general procedure, heating the reaction mixture to 245° C. for 90 min. Exactly 0.0206 mg of mesitylene (0.171 mmol) was used as an internal standard. ε-caprolactam was obtained in 89% yield.

### Table 2, Entry 14

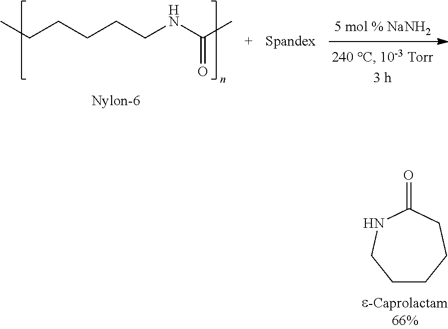

Exactly 50 mg of Nylon-6 powder (0.45 mmol), 5 mg of Spandex and 0.87 mg of NaNH₂ (5 mol %, 1.74 wt. %) were reacted according to the general procedure, heating the reaction mixture to 240° C. for 3 h. Exactly 23.0 mg of mesitylene (0.191 mmol) was used as an internal standard. ε-caprolactam was obtained in 66% yield. Table 2, entry 15

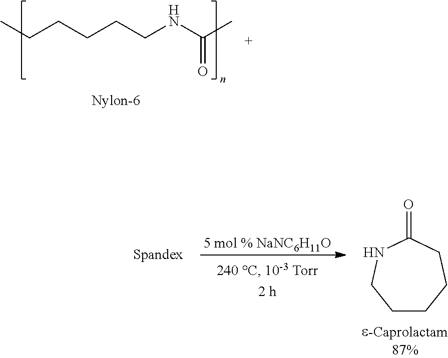

Exactly 90 mg of Nylon-6 powder (0.79 mmol), 10 mg of Spandex and 5.4 mg of NaNC₆H₁₁O (5 mol %, 6 wt. %) were reacted according to the general procedure, heating the reaction mixture to 240° C. for 2 h. Exactly 33.2 mg of mesitylene (0.276 mmol) was used as an internal standard. ε-caprolactam was obtained in 87% yield.

### Experimental Procedure for Continuous Depolymerization Reactions

Before Run 1: To a 100 mL oven-dried glass Schlenk reactor, 250 mg Nylon-6 powder mixed with 13.26 mg of LiNC₆H₁₁O was added in an Argon-filled glovebox. The reactor was sealed, and the polymer and catalyst were thoroughly mixed by stirring at room temperature for 5 min. The reactor was then evacuated to 10⁻³ Torr, sealed tightly, and heated to 240° C. for 20 min (Run 1). The reactor was cooled and placed back into the glovebox, and the caprolactam product was carefully scrapped off the cold wall of the reactor into a vial. The vial was weighed to determine the monomer recovery yield. Runs 2-3: After the removal of the product from the previous run, another 250 mg of fresh polymer was added to the bottom of the glass reactor. The reactor was then evacuated to 10⁻³ Torr, sealed tightly, and heated to 240° C. for 20 min (Run 2). The above procedure was repeated for Run 3 (30 min). Final run (Run 4): After the removal of the monomer product from Run 3, the reactor was removed from the glovebox without the addition of polymer. The reactor was then evacuated to 10³ Torr, sealed tightly, and heated to 240° C. for 6 h to ensure conversion of any remaining polymer. The reactor was then opened in air, exactly 41.4 mg of mesitylene was added as internal standard, and the yield for the final run was determined based on ¹HNMR analysis.

### Results and Discussion

This Example reports experimental results showing that the metal-organic and inorganic alkali metal superbases of the general structure of MXRₙ (M=Li, Na, K; X═H, C, N; R=alkyl, silyl, aryl, H) can efficiently depolymerize Nylon-6 to ε-caprolactam in quantitative yields (e.g., up to 99%), in short reaction times (e.g., as low as 10 min), at low temperatures (e.g., as low as 220° C.), and at low catalyst loadings (e.g., as low as 0.2 wt %), while functioning equally well under vacuum or inert atmosphere such as N₂ (Table 1). The results obtained using the super basic catalysts are compared under the same reaction conditions with those obtained using other catalysts used in existing techniques, such as K₂CO₃, Na₂CO₃, KOH, NaOH, and the NaOH—KOH eutectic mixture. The comparison shows that the present super basic catalysts exhibit instantaneous activity and provide significantly faster conversion rates at lower temperatures. These super basic catalysts have the advantage of being suitable for continuous operation, enabling a continuous feed of Nylon-6 during the reactions without experiencing a reduction in the reaction rate. Furthermore, the ε-caprolactam produced by the reaction can be successfully polymerized back to pristine Nylon-6.

In the first stage of the study, the performance of K₂CO₃, Na₂CO₃, KOH, NaOH, and the NaOH—KOH eutectic mixture was investigated under mild reaction conditions. However, in all cases, a significant amount of the starting Nylon-6 remained unreacted, resulting in yields of only 29%, 23%, and 3%, 3%, and 3% of ε-caprolactam, respectively. Note that the yield remained low despite increasing the reaction time to 6 hours and tripling the catalyst loading with respect to the previously reported loadings (Table 1, Entries 1-5).

After establishing that the catalysts as used in existing techniques had low activity under mild conditions, the activity of several super basic catalysts were tested. Surprisingly (given the relatively small ionic radii of alkali metal ions, e.g., as compared to lanthanide ions), using the MN(Si(CH₃)₃) 2 (M=K, Na, Li) catalysts gave a significant improvement in the rate of the reaction yielding selectively-caprolactam in 99%, 84%, and 99% yields, respectively, in 2 h at 240° C. (Table 1, Entries 6-8). Despite obtaining quantitative ε-caprolactam yields, the catalyst weight loadings used were relatively high (in the range of 7.5-8.8 wt %). Thus, it was investigated whether further enhancing the basicity of the catalysts would improve the overall performance of Nylon-6 depolymerization reactions.

To accomplish this, the super-basic amido MNH₂ and inorganic MH (M=Na, Li) catalysts were tested. These catalysts are commercially available on a ton scale, cost-effective, and have a simple ligand structure (NH₂ or H). These catalysts are also lightweight, requiring significantly lower weight loadings to achieve optimal results. However, these catalysts also have relatively low solubilities and high melting points (limiting mass transfer). Nevertheless, the results of Table 1 show that the catalysts have surprisingly high activities. More specifically, entries 9-14 in Table 1 show a systematic study where the effects of catalyst (NaNH₂) loading, reaction time, and temperature on the yield of ε-caprolactam were investigated. Note that the NaNH₂ catalyzed Nylon-6 depolymerization requires weight loading as low as 0.3 wt % (Table 1, Entry 9), yielding 81% ε-caprolactam after 1 h. Increasing the catalyst loading from 0.3 wt % to 1.7% and 3.4% increased the yield to 85% and 93%, respectively (Table 1, Entries 10 and 11). Increasing the loadings further to 6.9 wt % decreases the yield to 76% (Table 1, Entry 12). The reaction can operate identically under a static vacuum or an inert atmosphere at about 1 atm (N₂ or Ar; Table 1, Entry 13). Note that increasing the reaction temperature to 280° C. provides a 93% yield of ε-caprolactam after 10 min (Table 1 Entry 14). Interestingly, when NaH is used as a catalyst, an 83% yield of ε-caprolactam is produced after 1 h (Table 1, Entry 15).

Surprisingly, when lithium amide (LiNH₂), the smaller metal ion analog of sodium amide, is used, the conversion and yield were even further improved. This is surprising since the catalytic activity of depolymerization is expected to scale approximately with the ionic radius of the metal. The excellent performance of LiNH₂ was also unexpected as the melting point of LiNH₂ is 390° C., which is higher than the reaction temperatures and higher by 180° C. than NaNH₂ (mp=210° C.). For example, when 2 wt % of LiNH₂ is used, a 97% yield of ε-caprolactam was obtained after 2 h at 240° C. (Table 1, Entry 16). Furthermore, the reaction was essentially completed even when a lower catalyst loading is used (1 wt %) and a shorter reaction time (30 min) (Table 1, Entry 17). Increasing the reaction time to 1 h resulted in a slight yield increment to 94% yield (Table 1, Entry 18). Switching to an inert gas atmosphere (N₂ or Ar) yielded 87% ε-caprolactam (Table 1, Entry 19). Impressively, when the reaction temperature was raised to 280° C., a 99% yield of ε-caprolactam was obtained after only 10 min (Table 1, Entry 20). The catalyst loading was further decreased to 0.2 wt % without significantly affecting the reaction yield, producing 86% of ε-caprolactam after 2 h (Table 1, Entry 21). Increasing the reaction time to 4 h resulted in a higher yield of 95% (Table 1, Entry 22). Remarkably, the reaction proceeded smoothly at 220° C., the melting point of Nylon-6, affording a high yield of 87% after 4 h (Table 1, Entry 23). Note that at this temperature, Nylon-6 exhibits semi-solid properties, which hinders efficient mass transfer and mixing. Despite these challenges, the process was still efficient and yielded excellent results. In contrast, when the reaction was carried out with LiN(Si(CH₃)₃) 2 using identical conditions at the same mol % loadings of 1 mol %, the yield was noticeably lower, only reaching 62%, even after an extended reaction time of 6 h, (Table 1, Entry 24) which highlights once again the importance of the basicity of the catalyst. When 0.3 wt % of the lightest possible superbase LiH was used as the catalyst, an 86% yield is observed after 1 h (Table 1, Entry 25).

The catalytic process was also conducted on a larger scale and shown to be compatible with untreated Nylon-6 pellets and untreated colored Nylon-6 fishing nets. Thus, when 100 gr of air-stored untreated Nylon-6 pellets were reacted with 5 mol % (1.7 wt. %) of NaNH₂ at 250-260° C. for 45 min, a yield of 82% of caprolactam was obtained (Table 1, Entry 26). Interestingly, when an air-stored untreated colored Nylon-6 fishing net with a moisture content of 1.75 wt. % was used at a 100 gr scale under similar reaction conditions, an isolated yield mass of 90.57 gr was collected. The ε-caprolactam purity was 95.2%. The collected fraction was subjected to vacuum distillation at 10⁻¹ Torr at 140° C., yielding a 99% pure ε-caprolactam in 88% yield (Table 1, Entry 27). Additional experiments were carried out to examine the substituent effect on the alkali metal. When 5 mol % of LiNMe₂ was used, an 85% yield was observed after 1 h at 240° C. (Table 1, Entry 28), 9% lower compared to the reaction of LiNH₂ under identical conditions (Table 1, Entry 18) and 14% lower compared to LiN(Si(CH₃)₃) 2 (Table 1, Entry 8). Surprisingly, the catalysis is not limited only to N-containing catalysts, as when the organolithium reagent LiCH(Si(CH₃)₃) 2 was used, an 81% yield was obtained after 2 h (Table 1, Entry 29). This result was unexpected as organolithium reagents are known to nucleophilicity add to carbonyl groups to generate organo-substituted compounds such as oxygenates, and thus were expected to deactivate, rather than catalyze, the depolymerization of Nylon-6. In other words, in view of their high reactivity and low selectivity, it was expected that the organolithium reagents would be deactivated by side reactions, such as nucleophilic substitution. LiC₄H₉ (nBuLi) was also found to be catalytically active, yielding 77% yield after 2 h (Table 1, Entry 30). However, not all organometallic compounds exhibited catalytic activity. Thus, when NaCCH or Cp*Li were used 0% and 1.2% CPL yields were obtained after 2 h (Table 1, entries 31-32). Interestingly, exceptionally high yields were observed when testing catalysts with cyclic caprolactamate ligands on the metal with a general formula of MNC₆H₁₁O (M=Li, Na, K). Although some metal caprolactamates have been used as Nylon-6 polymerization catalysts in the conversion of caprolactam to Nylon-6, it was surprising that the present experiments showed that the MNC₆H₁₁O (M=Li, Na, K) could efficiently do the complete opposite—i.e., convert Nylon-6 back to caprolactam. Specifically, entries 33-38 in Table 1 show that using catalytic amounts (1-3 mol %) of MNC₆H₁₁O (M=Li, Na, K) affords 87-97% yield of caprolactam at 240° C. after 1 h.

TABLE 1
Alkali metal and anion screening of catalysts in Equation 1.^{a}

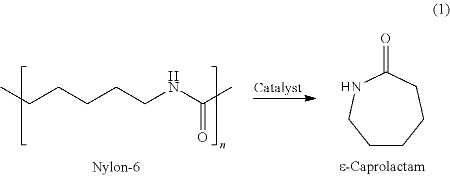

| | | Catalyst | Catalyst | | | | |
|---|---|---|---|---|---|---|---|
| | | loading | loading | | Temp. | | |
| Entry | Catalyst | (mol %) | (wt. %) | Time | (° C.) | Atmosphere | Yield (%)^{b} |
| 1 | K₂CO₃ | 5.0 | 6.1 | 6.0 h | 240 | 10⁻³ Torr | 29 |
| 2 | NaCO₃ | 5.0 | 4.7 | 6.0 h | 240 | 10⁻³ Torr | 23 |
| 3 | KOH | 5.0 | 2.5 | 6.0 h | 240 | 10⁻³ Torr | 3 |
| 4 | NaOH | 5.0 | 1.8 | 6.0 h | 240 | 10⁻³ Torr | 3 |
| 5 | KOH + NaOH (1:1) | 10.0 | 4.3 | 6.0 h | 240 | 10⁻³ Torr | 3 |
| 6 | KN(Si(CH₃)₃)₂ | 5.0 | 8.8 | 2.0 h | 240 | 10⁻³ Torr | >99 |
| 7 | NaN(Si(CH₃)₃)₂ | 5.0 | 8.0 | 2.0 h | 240 | 10⁻³ Torr | 94 |
| 8 | LiN(Si(CH₃)₃)₂ | 5.0 | 7.5 | 2.0 h | 240 | 10⁻³ Torr | 99 |
| 9 | NaNH₂ | 1.0 | 0.3 | 1.0 h | 240 | 10⁻³ Torr | 81 |
| 10 | NaNH₂ | 5.0 | 1.7 | 1.0 h | 240 | 10⁻³ Torr | 85 |
| 11 | NaNH₂ | 10 | 3.4 | 1.0 h | 240 | 10⁻³ Torr | 93 |
| 12 | NaNH₂ | 20 | 6.9 | 1.0 h | 240 | 10⁻³ Torr | 76 |
| 13 | NaNH₂ | 5.0 | 1.7 | 1.0 h | 240 | N₂ or Ar | 85 |
| 14 | NaNH₂ | 5.0 | 1.7 | 10 min | 280 | 10⁻³ Torr | 93 |
| 15 | NaH | 20 | 4.0 | 1.0 h | 240 | 10⁻³ Torr | 83 |
| 16 | LiNH₂ | 10 | 2.0 | 2.0 h | 240 | 10⁻³ Torr | 97 |
| 17 | LiNH₂ | 5.0 | 1.0 | 0.5 h | 240 | 10⁻³ Torr | 91 |
| 18 | LiNH₂ | 5.0 | 1.0 | 1.0 h | 240 | 10⁻³ Torr | 94 |
| 19 | LiNH₂ | 5.0 | 1.0 | 1.0 h | 240 | N₂ or Ar | 87 |
| 20 | LiNH₂ | 5.0 | 1.0 | 10 min | 280 | 10⁻³ Torr | 99 |
| 21 | LiNH₂ | 1.0 | 0.2 | 2.0 h | 240 | 10⁻³ Torr | 86 |
| 22 | LiNH₂ | 1.0 | 0.2 | 4.0 h | 240 | 10⁻³ Torr | 95 |
| 23 | LiNH₂ | 5.0 | 1.0 | 4.0 h | 220 | 10⁻³ Torr | 87 |
| 24 | LiN(Si(CH₃)₃)₂ | 1.0 | 1.5 | 6.0 h | 240 | 10⁻³ Torr | 62 |
| 25 | LiH | 5.0 | 0.3 | 1.0 h | 240 | 10⁻³ Torr | 86 |
| 26 | NaNH₂-Pellets^{c} | 5.6 | 1.95 | 45 min | 260-270 | 10⁻¹ Torr | 82^{d} |
| 27 | NaNH₂-purple | 5.0 | 1.7 | 50 min | 250-260 | 10⁻¹ Torr | 88^{d} |
| | fishing net^{e} | | | | | | |
| 28 | LiNMe₂ | 5.0 | 2.5 | 1.0 h | 240 | 10⁻³ Torr | 85 |
| 29 | LiCH(Si(CH₃)₃)₂ | 5.0 | 10.6 | 2.0 h | 240 | 10⁻³ Torr | 81 |
| 30 | LiC₄H₉ (nBuLi) | 5.0 | 2.8 | 2.0 h | 240 | 10⁻³ Torr | 77 |
| 31 | Cp*Li | 5.0 | 6.3 | 2.0 h | 240 | 10⁻³ Torr | 1.2 |
| 32 | NaCCH | 5.0 | 2.1 | 2.0 h | 240 | 10⁻³ Torr | 0 |
| 33 | LiNC₆H₁₁O | 3.0 | 3.2 | 1.0 h | 240 | 10⁻³ Torr | 93 |
| 34 | LiNC₆H₁₁O^{e} | 1.1 | 1.2 | 1.0 h | 240 | 10⁻³ Torr | 89 |
| 35 | NaNC₆H₁₁O | 3.0 | 3.6 | 1.0 h | 240 | 10⁻³ Torr | 97 |
| 36 | NaNC₆H₁₁O^{e} | 1.0 | 1.2 | 1.0 h | 240 | 10⁻³ Torr | 94 |
| 37 | KNC₆H₁₁O | 3.0 | 4.0 | 1.0 h | 240 | 10⁻³ Torr | 94 |
| 38 | KNC₆H₁₁O^{e} | 1.0 | 1.4 | 10 min | 280 | 10⁻³ Torr | 87 |

^{a}General depolymerization conditions: static vacuum (10⁻³ Torr), 100 mg of Nylon-6 powder in 50
mL Schlenk flask, unless otherwise stated.
^{b}Yields determined by ¹H NMR with a mesitylene internal standard.
^{c}3 L flask.
^{d}Isolated yields.
^{e}100 mL Schlenk flask.

Next, experiments were conducted to determine whether the present catalysts could depolymerize Nylon-6 in the presence of other polymers such as polyamide 12 (Nylon 12), Polyamide 66 (Nylon 66), Polyethylene (PE), polyethylene-co-1-octene (PECO), isotactic polypropylene (i-PP), polyethylene terephthalate (PET), polyethylene glycol (PEG), poly-paraphenylene terephthalamide (Kevlar), Polytetrafluoroethylene (PTFE), Polyurethane-polyurea copolymer (Spandex), as shown in Equation 2. Surprisingly, when Nylon 6 was mixed with other polyamides, such as Nylon 12 or Nylon 66, the high selectivity and activity toward converting Nylon 6 to caprolactam were maintained despite having potentially competing amide groups in the mixture. Thus, when a 1:1 mixture of Nylon 6 and Nylon 12 was heated in the presence of 5 mol % of NaNC₆H₁₁O or NaNH₂, caprolactam was obtained in 95% and 73% yields, respectively (Table 2, entries 1-2). When a 1:1 mixture of Nylon 6 and Nylon 66 was used, a 81% yield of caprolactam was obtained (Table 2, Entry 3). In the cases when a quantitative conversion of Nylon-6 to caprolactam was observed, the other polyamide could be recovered unaltered, offering a unique solvent-free way of chemically separating different polyamides.

The process was also compatible with various types of plastic mixtures. Thus, when Nylon 6 was mixed with polyolefins such as polyethylene (PE), isotactic polypropylene (i-PP), or polyethylene-co-1-octene (PECO), caprolactam was quantitatively recovered in 78-90% yields (Table 2, entries 4-8) leaving at the bottom of the reactor an unaltered polyolefin. The process was also surprisingly compatible with oxygen-rich polymers such as polyesters and polyether. In the case of polyethylene terephthalate (PET), yields in the range of 52-75% of caprolactam were obtained (Table 2, entries 9-10), and with polyethylene glycol (PEG), a yield of 81% was observed (Table 2, entry 11).

The process also worked in the presence of post-consumer high melting point polymers such as Kevlar fibers taken from a glove and Teflon tape (PTFE). In the case of Kevlar due to challenging mass transfer issues and lack of stirring, modest caprolactam yields of 66% was observed (Table 2, entries 12). In the case of Teflon, an 89% yield of caprolactam was observed (Table 2, Entry 13). Interestingly, the process can also work in the presence of polyurethanes such as Spandex, a polymer that is commonly blended with Nylon-6, yielding 66-87% caprolactam (Table 2, entries 14-15).

TABLE 2
Nylon-6 depolymerization in the presence of others polymers as shown in Equation 2.^{a} 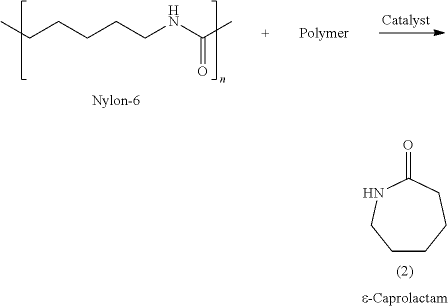
Polymer = Nylon 12, Nylon 66, PE, PECO, i-PP, PET, PEG, Kevlar, PTFE, Spandex

| | | | Catalyst loading | Time | Temperature | |
|---|---|---|---|---|---|---|
| Entry | Catalyst | Plastics | (mol %) | (min) | (° C.) | Yield (%)^{b} |
| 1 | NaNC₆H₁₁O | Nylon 6: Nylon 12 | 5 | 180 | 240 | 95 |
| 2 | NaNH₂ | Nylon 6: Nylon 12 | 5 | 180 | 240 | 73 |
| 3 | NaNC₆H₁₁O | Nylon 6: Nylon 66 | 5 | 360 | 260 | 81 |
| 4 | NaNC₆H₁₁O | Nylon 6: PE | 5 | 180 | 240 | 88 |
| 5 | NaNH₂ | Nylon 6: PE ^{c} | 5 | 180 | 280 | 84 |
| 6 | NaNC₆H₁₁O | Nylon 6: i-PP | 5 | 180 | 240 | 85 |
| 7 | NaNH₂ | Nylon 6: i-PP | 5 | 180 | 240 | 90 |
| 8 | NaNC₆H₁₁O | Nylon 6: PECO | 5 | 180 | 240 | 78 |
| 9 | NaNC₆H₁₁O | Nylon 6: PET ^{d,e} | 5.7 | 90 | 240 | 75 |
| 10 | NaNH₂ | Nylon 6: PET ^{f} | 5 | 180 | 280 | 52 |
| 11 | NaNH₂ | Nylon 6: PEG ^{e,g} | 5.8 | 20 | 280 | 81 |
| 12 | NaNC₆H₁₁O | Nylon 6: Kevlar | 5 | 180 | 240 | 66 |
| 13 | NaNH₂ | Nylon 6: PTFE ^{c,e} | 5 | 90 | 245 | 89 |
| 14 | NaNH₂ | Nylon 6: spandex ^{f} | 5 | 180 | 240 | 66 |
| 15 | NaNC₆H₁₁O | Nylon 6: spandex ^{e,h} | 5 | 120 | 240 | 87 |

^{a}General depolymerization conditions: static vacuum (10⁻³ Torr), 50 mg of Nylon-6 powder, and 50 mg
of a different polymer in a 50 mL Schlenk flask, unless otherwise stated.
^{b}Yields determined by ¹H NMR with mesitylene internal standard.
^{c} The plastics mixture Nylon-6: other polymer ratio was 3:1.
^{d} The plastics mixture Nylon-6: other polymer ratio was 7:3.
^{e} 100 mL Schlenk flask.
^{f} The plastics mixture Nylon-6: other polymer ratio was 10:1.
^{g} The plastics mixture Nylon-6: other polymer ratio was 5:1.
^{h} The plastics mixture Nylon-6: other polymer ratio was 9:1.

Finally, to test the recyclability of the alkali superbase catalysts, a multi-batch simulated continuous depolymerization experiment was performed in a larger-scale reactor (100 mL flask) using catalyst LiNC₆H₁₁O. Before each run, the caprolactam collected from the previous run was removed and weighed, and 250 mg of fresh Nylon-6 was added to the reactor containing the same catalyst LiNC₆H₁₁O charge. The results demonstrated that the catalyst LiNC₆H₁₁O retained a high level of activity through each run. The final (4th) run was performed without the addition of fresh Nylon, providing an overall Nylon-6 yield of 79.3%. Results are shown in the Table 3 below:

TABLE 3
Multi-batch Simulated Continuous Nylon-6 Depolymerization.

| | 1^{st} Run | | 2^{nd} Run | | 3^{rd} Run | | 4^{th} Run | |
|---|---|---|---|---|---|---|---|---|
| Nylon-6 | +250 | mg | +250 | mg | +250 | mg | 0 | |
| LiNC₆H₁₁O | +13.26 | mg | 0 | | 0 | | 0 | |
| Reaction time | 20 | min | 20 | min | 30 | min | 6 | h |
| Isolated yield | 170 | mg | 226 | mg | 124 | mg | 75 | mg |

The word “illustrative” is used herein to mean serving as an example, instance, or illustration. Any aspect or design described herein as “illustrative” is not necessarily to be construed as preferred or advantageous over other aspects or designs. Further, for the purposes of this disclosure and unless otherwise specified, “a” or “an” means “one or more.”

If not already included, all numeric values of parameters in the present disclosure are proceeded by the term “about” which means approximately. This encompasses those variations inherent to the measurement of the relevant parameter as understood by those of ordinary skill in the art. This also encompasses the exact value of the disclosed numeric value and values that round to the disclosed numeric value.

The foregoing description of illustrative embodiments of the disclosure has been presented for purposes of illustration and of description. It is not intended to be exhaustive or to limit the disclosure to the precise form disclosed, and modifications and variations are possible in light of the above teachings or may be acquired from practice of the disclosure. The embodiments were chosen and described in order to explain the principles of the disclosure and as practical applications of the disclosure to enable one skilled in the art to utilize the disclosure in various embodiments and with various modifications as suited to the particular use contemplated. It is intended that the scope of the disclosure be defined by the claims appended hereto and their equivalents.

1. A method for depolymerizing a polyamide, the method comprising: combining a polyamide and an alkali metal catalyst to depolymerize the polyamide to a product, wherein the alkali metal catalyst is selected from those having a formula MXRₙ, wherein M is an alkali metal; X is N, H, C, Si, or Sn; R is independently selected from hydrogen, alkyl, silyl, and aryl; and n is from 2 to 0.

2. The method of claim 1, wherein M is selected from Na, Li, K, Cs, and a mixture thereof.

3. The method of claim 1, wherein M is selected from Na, Li, K, Cs and a mixture thereof; X is N or H; and R is independently selected from hydrogen, alkyl, and silyl.

4. The method of claim 1, wherein M is selected from Na, Li, K, Cs and a mixture thereof; X is N; R is hydrogen; and n is 2.

5. The method of claim 4, wherein M is Na.

6. The method of claim 1, wherein M is selected from Na, Li, K, Cs and a mixture thereof; X is H; and n is 0.

7. The method of claim 6, wherein M is Na.

8. (canceled)

9. The method of claim 3, wherein X is H, R is the alkyl, the alkyl is a substituted cycloalkyl, and n is 1.

10. The method of claim 9, wherein M is Na.

11. The method of claim 1, wherein the polyamide is a polymerization product of a monomer selected from ε-caprolactam, 2-pyrrolidone, 2-piperidone, enantholactam, capryllactam, pelargolactam, azacycloundecan-2-one, azacyclotridecan-2-one, and combinations thereof.

12. The method of claim 1, wherein the polyamide is selected from poly(hexano-6-lactam) (Nylon-6), poly(2-pyrrolidinone) (Nylon-4), poly(2-piperidone) (Nylon-5), polyenanthamide (Nylon-7), polycapryllactam (Nylon-8), poly(9-aminononanoic acid (Nylon-9), poly(10-aminodecanoic acid) (Nylon-10), poly(11-aminoundecanoic acid) (Nylon-11), poly(dodecano-12-lactam) (Nylon-12), poly[imino(1,6-dioxo hexamethylene)imino hexamethylene] (Nylon-66), poly[imino(1,6-dioxohexamethylene) iminotetramethylene] (Nylon-46), and combinations thereof.

13. The method of claim 1, wherein the product comprises a monomer from which the polyamide was formed.

14. The method of claim 13, wherein the monomer is a cyclic amide.

15. The method of claim 1, wherein the method is carried out in an absence of a liquid medium.

16. The method of claim 1, wherein the polyamide and the alkali metal catalyst form a reaction mixture consisting of the polyamide and the alkali catalyst.

17. The method of claim 1, wherein the method is carried out using a continuous flow reactor system.

18. The method of claim 1, wherein the method further comprises recovering the product from a reaction mixture comprising the polyamide and the alkali metal catalyst, wherein the product comprises a monomer from which the polyamide was formed.

19. The method of claim 3, wherein the method is carried out using a temperature of 240° C. or less, a time of 4 hours or less, a loading of the alkali metal catalyst of 10 mol % or less, and a pressure of 10⁻³ Torr or less, and the method provides a yield of the product of at least 90%.

20. The method of claim 19, wherein the polyamide comprises poly(hexano-6-lactam) (Nylon-6).

21. The method of claim 20, wherein the product comprises a monomer from which the polyamide was formed.
